(19) United States
(12) Patent Application Publication
(10) Pub. No.: US 2003/0032000 A1
(43) Pub. Date: Feb. 13, 2003

(54) Method for analyzing cellular events

(75) Inventors: Vivian F. Liu, San Francisco, CA (US); Prasanthi Bhagavatula, Palo Alto, CA (US); Uyen T. Do, San Jose, CA (US); John J. Hefti, San Francisco, CA (US)

Correspondence Address: RICHARD L. NEELEY, SIGNATURE BIOSCIENCE, INC., 475 BRANNAN STREET, SAN FRANCISCO, CA, 94107, US

(73) Assignee: Signature BioScience Inc., Hayward, CA

(21) Appl. No.: 09/929,513

(22) Filed: Aug. 13, 2001

(51) Int. Cl. C12Q 1/00; C12Q 1/68

(52) U.S. Cl. 435/4; 205/777.5; 435/6

(57) ABSTRACT

The present invention makes it possible to detect cellular activity in a sensitive and efficient manner without the use of labels and without knowing specifically what activity is being detected, although detection of specific activity is possible. The assay comprises detecting cellular activity by monitoring a change in a cellular system, comprising coupling an electromagnetic test signal in a specified frequency range to a sample in which a cellular event is being detected, whereby the sample interacts with and modulates the test signal to produce a modulated test signal; detecting the modulated test signal; and analyzing the modulated test signal to detect said cellular event. As such, the present invention is particularly useful in the detection of cellular activity induced by the presence of a test substance in the medium in which a cell is located and provides a number of advantages for lead optimization in the drug discovery field.

### BACKGROUND OF THE INVENTION

Recent developments in the laboratory of the present inventors have enhanced the ability of researchers to detect molecular events in solution and in real time without requiring molecular labels or extra process steps. The first developments involved a molecular binding layer or region used to capture potential ligands, with the molecular binding layer or region being electromagnetically coupled to a transmission line that carried the appropriate electromagnetic signal. See, for example, U.S. application Ser. No. 09/243194, filed Feb. 2, 1999, and U.S. application Ser. No. 09/365578, filed Aug. 8, 1999. Some embodiments of the earliest development used a signal that did not penetrate deeply into the overlying solution, so that binding interactions could be easily detected regardless of the content of the overlying solution, which was essentially invisible under the experimental conditions. Other developments directly detected molecular events in solution, using a signal that penetrates into the solution. See, for example, U.S. patent application Ser. No. 09/687,456, filed Oct. 13, 2000. In either case, these new techniques make it possible to detect binding interactions without washing or other separation steps.

One of the goals of the present technological developments is to apply technology developed in the detection of molecular events, which requires great sensitivity of instrumentation and tight control of samples and detection techniques, to cellular events. In particular, it is desirable to detect changes in cellular activity as the result of the addition of a test compound (often a potential pharmaceutical compound) to a cell medium in order to assess the potential effects that the test compound may have on the activity of living cells.

### SUMMARY OF THE INVENTION

Earlier patent applications from the laboratories of the present inventors have dealt with an improvement in measurement techniques for detecting changes in the dielectric properties of molecular events. These earlier applications describe many of the general principles used with the present invention. See, for example, the patent applications listed at the end of the current specification. The present invention, while also being generally directed to detection of molecular events, is different in that it focuses on cellular events, notably the detection of a change in cellular activity as a result of the addition of a test substance to the medium (sample) in which the cells are located. Such detection of a change in cellular activity, coupled with analysis of that activity change by detection of an electromagnetic signal coupled to the cell, provides much useful information in the drug discovery field, as it allows assignment of greater pharmaceutical relevance of test compounds than is possible with simple detection of a molecular binding event in the absence of cells, by identifying both desirable and undesirable cellular activity resulting from contact of cells with potential pharmaceutical products. As an example, it can be used to detect and distinguish agonist and antagonist activity.

The nature and advantages of the present invention will be better understood with reference to the following drawings and detailed description.

### BRIEF DESCRIPTION OF THE DRAWINGS

FIG. 1 illustrates a bioassay test system in accordance with one embodiment of the detection system.

FIG. 2 illustrates a first embodiment of a biosensor in accordance with the present invention.

FIG. 3 illustrates a second embodiment of the biosensor in accordance with the present invention.

FIG. 4A illustrates a third embodiment of the biosensor in accordance with the present invention.

FIG. 4B illustrates a fourth embodiment of the biosensor in accordance with the present invention.

FIG. 5 illustrates a coaxial biosensor integrated with a fluidic transport system in accordance with one embodiment of the present invention.

FIG. 6 illustrates a bioassay test system in which a flow tube is used to supply the sample to a coaxial probe in accordance with the present invention.

FIG. 7 illustrates a flow cell for use with the waveguide magic-t detector shown in FIG. 4 in accordance with the present invention.

FIG. 8A illustrates a simplified block diagram of a computer system 810 operable to execute a software program designed to perform each of the described methods.

FIG. 8B illustrates the internal architecture of the computer system 810.

In addition, a number of copies of presentation slides are included as part of this specification. The material shown in these presentation slides is also described in the detailed description below.

### DESCRIPTION OF THE SPECIFIC EMBODIMENTS

Table of Contents

I General Overview

II Definition of Terms

III Signal Analysis Techniques

IV Exemplary Molecular Detection Systems

V Exemplary Applications

VI Software Implementation

VII. Experiments

I. General Overview

The present invention makes it possible to detect cellular activity in a sensitive and efficient manner without the use of labels and even without knowing specifically what activity is being detected, although detection of specific activity is possible as described below. As such, the present invention is particularly useful in the detection of cellular activity induced by the presence of a test substance in the medium in which a cell is located and provides a number of advantages for lead optimization in the drug discovery field.

II. Definition of Terms

The following definitions are grouped under subheadings for ease of reference. Inclusion of a definition under one subheading should not be taken as an indication that the definition is limited to structures and events common to that subheading. Any intended limitations on the definitions will be provided by the definitions themselves.

Chemistry and Biologics

As used herein, the term “molecular event” refers to the interaction of a molecule of interest with another molecule (e.g., molecular binding) and to all structural properties of molecules of interest. Structural molecular properties include the presence of specific molecular substructures (such as alpha helix regions, beta sheets, immunoglobulin domains, and other types of molecular substructures), as well as how the molecule changes its overall physical structure via interaction with other molecules (such as by bending or folding motions), including the molecule's interaction with its own salvation shell while in solution. The simple presence of a molecule of interest in the region where detection/analysis is taking place is not considered to be a “molecular event,” but is referred to as a “presence.”

Examples of molecular binding events are (1) simple, non-covalent binding, such as occurs between a ligand and its antiligand, and (2) temporary covalent bond formation, such as often occurs when an enzyme is reacting with its substrate. More specific examples of binding events of interest include, but are not limited to, ligand/receptor, antigen/antibody, enzyme/substrate, DNA/DNA, DNA/RNA, RNA/RNA, nucleic acid mismatches, complementary nucleic acids and nucleic acid/proteins. Binding events can occur as primary, secondary, or higher order binding events. A primary binding event is defined as a first molecule binding (specifically or non-specifically) to an entity of any type, whether an independent molecule or a material that is part of a first surface, typically a surface within the detection region, to form a first molecular interaction complex. A secondary binding event is defined as a second molecule binding (specifically or non-specifically) to the first molecular interaction complex. A tertiary binding event is defined as a third molecule binding (specifically or non-specifically) to the second molecular interaction complex, and so on for higher order binding events.

Examples of relevant molecular structures are the presence of a physical substructure (e.g., presence of an alpha helix, a beta sheet, a catalytic active site, a binding region, or a seven-trans-membrane protein structure in a molecule) or a structure relating to some functional capability (e.g., ability to function as an antibody, to transport a particular ligand, to function as an ion channel (or component thereof), or to function as a signal transducer). Molecular structure is typically detected by comparing the signal obtained from a molecule of unknown structure and/or function to the signal obtained from a molecule of known structure and/or function. Molecular binding events are typically detected by comparing the signal obtained from a sample containing one of the potential binding partners (or the signals from two individual samples, each containing one of the potential binding partners) to the signal obtained from a sample containing both potential binding partners.

The term “cellular event” refers in a similar manner to reactions and structural rearrangements occurring as a result of the activity of a living cell (which includes cell death). Examples of cellular events include opening and closing of ion channels, leakage of cell contents, passage of material across a membrane (whether by passive or active transport), activation and inactivation of cellular processes, as well as all other functions of living cells. Cellular events are commonly detected by comparing modulated signals obtained from two cells (or collection of cells) that differ in some fashion, for example by being in different environments (e.g., the effect of heat or an added cell stimulant) or that have different genetic structures (e.g., a normal versus a mutated or genetically modified cell). Morpholic changes are also cellular events.

The same bioassay systems can be used for molecular and cellular events, differing only in the biological needs of the cells versus the molecules being tested. Accordingly, this specification often refers simply to molecular events (the more difficult of the two measurements under most circumstances) for simplicity, in order to avoid the awkwardness of continually referring to “molecular and/or cellular” events, detection, sample handling, etc., when referring to an apparatus that can be used to detect either molecular events or cellular events. When appropriate for discussion of a particular event, the event will be described as, for example, a cellular event, a molecular binding event, or a molecular structure determination.

When a molecular event (e.g., binding of a potential drug with a receptor) is being detected in a biological sample capable of undergoing biological functions (e.g., a cell or a cell-free enzyme system), the molecular event can be amplified by the biological function and, if desired to increase sensitivity, the change resulting from the function can be detected rather than the molecular event itself. Examples of detectable amplified signals include the permittivity change of a cell resulting from the opening or closing of an ion channel when a molecular binding event occurs and a physiological reaction (e.g., synthesis of a protein) of a cell when a drug interacts with a cellular receptor. When working with cells, such binding event detection can be referred to as detection of a “cellular molecular event” (as opposed to a “non-cellular molecular event,” which is one that occurs in the absence of cells). Similar language can be used to describe cell-free enzyme-system molecular events.

As used herein, the term “analyte” refers to a molecular or cellular entity whose presence, structure, binding ability, etc., is being detected or analyzed. Suitable analytes for practice of this invention include, but are not limited to antibodies, antigens, nucleic acids (e.g. natural or synthetic DNA, RNA, gDNA, cDNA, mRNA, tRNA), lectins, sugars, glycoproteins, receptors and their cognate ligand (e.g. growth factors and their associated receptors, cytokines and their associated receptors, signaling molecules and their receptors), small molecules such as existing pharmaceuticals and drug candidates (either from natural products or synthetic analogues developed and stored in combinatorial libraries), metabolites, drugs of abuse and their metabolic by-products, co-factors such as vitamins and other naturally occurring and synthetic compounds, oxygen and other gases found in physiologic fluids, cells, cellular constituents, cell membranes and associated structures, other natural products found in plant and animal sources, and other partially or completely synthetic products.

The word “ligand” is commonly used herein to refer to any molecule for which there exists another molecule (i.e. an “antiligand”) that binds to the ligand, owing to a favorable (i.e., negative) change in free energy upon contact between the ligand and antiligand. There is no limit on the size of the interacting substances, so that a ligand (or an antiligand) in this broad sense can consist of either an individual molecule or a larger, organized group of molecules, such as would be presented by a cell, cell membrane, organelle, or synthetic analogue thereof. As used herein, “ligand” and “antiligand” both have this broad sense and can be used interchangeably. However, it is recognized that there is a general tendency in the field of biology to use the word “ligand” to refer to the smaller of the two binding partners that interact with each other, and this convention is followed whenever possible.

As used herein, the term “ligand/antiligand complex” refers to the ligand bound to the antiligand. The binding can be specific or non-specific, and the interacting ligand/antiligand complex are typically bonded to each other through noncovalent forces such as hydrogen bonds, Van der Waals interactions, or other types of molecular interactions.

As used herein, the term “specifically binds,” when referring to a protein, nucleic acid, or other binding partner as described herein, refers to a binding reaction which is selective for the ligand of interest in a heterogeneous population of potential ligands. Thus, under designated conditions (e.g. immunoassay conditions in the case of an antibody), the specified antiligand binds to its particular “target” and does not bind in an indistinguishable amount to other potential ligands present in the sample. For example, a cell surface receptor for a hormonal signal (e.g., the estrogen receptor) will selectively bind to a specific hormone (e.g., estradiol), even in the presence of other molecules of similar structure (such as other steroidal hormones, even similar steroids such as estriol). Similarly, nucleic acid sequences that are completely complementary will hybridize to one another under preselected conditions such that other nucleic acids, even those different in sequence at the position of a single nucleotide, do not hybridize.

Although measurements described herein are often made on individual molecules or pairs of molecules in solution, at times the method of the invention can be applied to situations in which one of the members of a binding pair is immobilized on a surface while test compounds in solution contact the immobilized molecule (individually, in a mixture, or sequentially). As used herein, when one member of a binding pair is immobilized, the term “antiligand” is usually used to refer to the molecule immobilized on the surface. The antiligand, for example, can be an antibody and the ligand can be a molecule such as an antigen that binds specifically to the antibody. In the event that an antigen is bound to the surface and the antibody is the molecule being detected, for the purposes of this document the antibody can be considered to be the ligand and the antigen considered to be the antiligand. Additionally, once an antiligand has bound to a ligand, the resulting antiligand/ligand complex can be considered an antiligand for the purposes of subsequent binding.

As used herein, the terms “molecule” refers to a biological or chemical entity that exists in the form of a chemical molecule or molecules, as opposed to salts or other non-molecular forms of matter. Many molecules are of the type referred to as organic molecules (compounds containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, receptors, antibodies, and lipids. When appropriate, one or more of these more descriptive terms (many of which, such as “protein,” themselves describe overlapping groups of compounds) will be used herein because of application of the method to a subgroup of molecules, without detracting from the intent to have such compounds be representative of both the general class “molecules” and the named subclass, such as proteins. When used in its most general meaning, a “molecule” also includes bound complexes of individual molecules, such as those described below. An ionic bond can be present in a primarily covalently bound molecule (such as in a salt of a carboxylic acid or a protein with a metal ion bound to its amino acid residues), and such molecules are still considered to be molecular structures. Of course, it is also possible that salts (e.g., sodium chloride) will be present in the sample that contains a molecular structure, and the presence of such salts does not detract from the practice of the invention. Such salts will participate in the overall dielectric response, but a molecular binding event or property can be detected in their presence.

As used herein, the terms “binding partners,” “ligand/antiligand,” or “ligand/antiligand complex” refers to pairs (or larger groups; see below) of molecules that specifically contact (e.g. bind to) each other to form a bound complex. Such a pair or other grouping typically consists of two or more molecules that are interacting with each other, usually by the formation of non-covalent bonds (such as dipole-dipole interactions, hydrogen bonding, or van der Waals interactions). The time of interaction (sometimes referred to as the on-off time) can vary considerably, even for molecules that have similar binding affinities, as is well known in the art. Examples include antibody-antigen, lectin-carbohydrate, nucleic acid-nucleic acid, and biotin-avidin pairs. Biological binding partners need not be limited to pairs of single molecules. Thus, for example, a single ligand can be bound by the coordinated action of two or more anti-ligands, or a first antigen/antibody pair can be bound by a second antibody that is specific for the first antibody. Binding can occur with all binding components in solution or with one (or more) of the components attached to a surface and can include complex binding that involves the serial or simultaneous binding of three or more separate molecular entities. Examples of complex binding include GPCR-ligand binding, followed by GPCR/G-protein binding; nuclear receptor/cofactor/ligand/DNA binding; or a binding complex including chaperone proteins, along with a small-molecule ligand. Other examples will be readily apparent to those skilled in the art.

As used herein, the terms “isolated,” “purified,” and “biologically pure” refer to material which is substantially or essentially free from components that normally accompany it as found in its native state.

As used herein, the term “nucleic acid” refers to a deoxyribonucleotide or ribonucleotide polymer in either single- or double-stranded form, and, unless otherwise limited, encompasses such polymers that contain one or more analogs of natural nucleotides that can hybridize in a similar manner to naturally occurring nucleotides.

As used herein, the terms “polypeptide,” “peptide,” and “protein” are generally used interchangeably to refer to a polymer of amino acid residues. These terms do not appear to have a consistent use in the art in reference to the size of molecules, although “polypeptide” is often used without regard to size, while “peptides” are smaller than “proteins.” Proteins are generally considered to be more complex than simple peptides and often contain material other than amino acids, such as polysaccharide chains. All of these terms apply to polymers containing amino acids in which one or more amino acid residue or peptide bond is an artificial chemical analogue of a corresponding naturally occurring amino acid or bond, as well as to naturally occurring amino acid polymers.

As used herein, the term “antibody” refers to a protein consisting of one or more polypeptide chains substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface”. This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987).

As used herein, the terms “immunological binding” and “immunological binding properties” refer to the non-covalent interactions of the type that occur between an immunoglobulin molecule and an antigen for which the immunoglobulin is specific.

As used herein, the term “enzyme” refers to a protein that acts as a catalyst and reduces the activation energy of a chemical reaction occurring between other compounds or of a chemical reaction in which one compound is broken apart into smaller compounds. The compounds that undergo the reaction under the influence of the enzyme are referred to as “substrates.” The enzyme is not a starting material or final product in the reaction, but is unchanged after the reaction is completed.

As used herein, the terms “molecular binding layer” or “MBL” refers to a layer having at least one molecular structure (e.g., an analyte, antiligand, or a ligand/antiligand pair) that is electromagnetically coupled to the signal path. The MBL is typically formed on a fixed surface in the detection region, although mobile surfaces, such as beads or cells, can easily be used along with appropriate fluid movement controls. The molecular binding layer can consist of one or more ligands, antiligands, ligand/antiligand complexes, linkers, matrices of polymers and other materials, or other molecular structures described herein. Further, the molecular binding layer can be extremely diverse and can include one or more components, including matrix layers and/or insulating layers, that have one or more linking groups. The MBL can be electromagnetically coupled to the signal path either via a direct or indirect physical connection or when the ligand is located proximate to, but physically separated from, the signal path. The MBL can be formed on a derivatized surface, such as one having thiol linkers formed from biotinylated metals, all in accordance with standard practice in the art. Sometimes the term “molecular binding region” or “MBR” is used instead of MBL, particularly in cases where the geometry is more complex than a simple layer.

As used herein, the term “linking group” or “linker” refers to a chemical structure used to attach any two components to each other, often on the bioassay device. The linking groups thus have a first binding portion that binds to one component, such as the conductive surface, and a second binding portion that binds to another component, such as the matrix or the antiligand.

Mechanics and Sample Handling

As used herein, the term “solution” refers to the resulting mixture formed from a first material (the “solvent,” which forms the bulk of the solution) in which a second material (the “solute”, such as a target ligand) resides primarily as separate molecules rather than as aggregates of molecules. Solutions can exist in any of the solid, liquid or gaseous states. Solid solutions can be formed from “solvents” made of naturally occurring or synthetic molecules, including carbohydrates, proteins, and oligonucleotides, or of organic polymeric materials, such as nylon, rayon, dacron, polypropylene, teflon, neoprene, and delrin. Liquid solutions include those containing an aqueous, organic or other liquid solvent, including gels, emulsions, and other viscous materials formed from liquids mixed with other substances. Exemplary liquid solutions include those formed from celluloses, dextran derivatives, aqueous solution of d-PBS, Tris buffers, deionized water, blood, physiological buffer, cerebrospinal fluid, urine, saliva, water, and organic solvents, such as ethers or alcohols. Gaseous solutions can consist of organic molecules as gases or vapors in air, nitrogen, hydrogen, or other gaseous solvents. The word “solution” is used herein in many cases to refer to a mixture containing a target ligand and/or antiligand that is being applied to a molecular binding surface. Another example of a solution is the sample that is being analyzed. As previously indicated, liquid solutions, particularly aqueous ones, are preferred for the practice of the invention.

As used herein, the term “test sample” refers to the material being investigated (the analyte) and the medium/buffer in which the analyte is found. The medium or buffer can included solid, liquid or gaseous phase materials; the principal component of most physiological media/buffers is water. Solid phase media can be comprised of naturally occurring or synthetic molecules including carbohydrates, proteins, oligonucleotides, SiO₂, GaAs, Au, or alternatively, any organic polymeric material, such as Nylon®, Rayon®, Dacron®, polypropylene, Teflon®, neoprene, delrin or the like. Liquid phase media include those containing an aqueous, organic or other primary components, gels, gases, and emulsions. Exemplary media include celluloses, dextran derivatives, aqueous solutions of d-PBS, Tris, deionized water, blood, cerebrospinal fluid, urine, saliva, water, and organic solvents.

As used herein, a “biological sample” is a sample of biological tissue or fluid that, in a healthy and/or pathological state, is to be assayed for the structure(s) or event(s) of interest. Such biological samples include, but are not limited to, sputum, amniotic fluid, blood, blood cells (e.g., white cells), tissue or fine needle biopsy samples, urine, peritoneal fluid, pleural fluid, and cells from any of these sources. Biological samples also include cells grown in cultures, both mammalian and others. Biological samples further include sections of tissues such as frozen sections taken for histological purposes. Although a biological sample is often taken from a human patient, the meaning is not so limited. The same assays can be used to detect a molecular event of interest in samples from any mammal, such as dogs, cats, sheep, cattle, and pigs, as well as samples from other animal species (e.g., birds, such as chickens or turkey) and plants (e.g., ornamental plants and plants used as foods, such as corn or wheat). The biological sample can be pretreated as necessary by dilution in an appropriate transporting medium solution or concentrated, if desired, and is still referred to as a “biological sample.” Any of a number of standard aqueous transporting medium solutions, employing one of a variety of transporting media, such as phosphate, Tris, or the like, preferably at physiological pH can be used. As with biological samples, pretreatment of a more general sample (by dilution, extraction, etc.) once it is obtained from a source material do not prevent the material from being referred to as a sample.

As used herein, the term “fluid reservoir” refers to any location, without regard to physical size or shape, in which the sample fluid is retained prior or subsequent to application of the sample fluid across the detection region. “Fluid reservoir” can refer to a fluid droplet or layer formed on a flat surface and maintained at that location by inertia and/or surface tension. Such arrangements are sometimes used in various “chip” designs commonly used in genomics in which a sample fluid is washed across the surface of a chip that has specific molecular probes (usually DNA fragments of know sequence) attached at known locations on the surface. The “fluid reservoir,” however, can be and often is contained within physical walls that restrain movement of the fluid, such as vertical walls that constrain gravitational spreading (as in the side walls of test tube or of the well of a microtitre plate), completely surrounding walls (as in a sealed container), or partially surrounding walls that direct and/or permit motion in a limited number of directions (such as the walls of a tube or other channel). The last of these named possibilities is often referred to herein as a “fluid channel” and occurs commonly in situations were a fluid is being moved from one location to another (such as in a microfluidics chip) to allow interaction with other samples and/or solutions containing reagents or to allow multiple samples to be transported past a single detection region.

Electronics

As used herein, the term “signal path” refers to a transmission medium that supports the propagation of an electromagnetic signal at the desired frequency of operation. In one embodiment, the transmission path consists of a signal plane/ground plane/dielectric substrate structure capable of supporting a transverse electromagnetic (TEM) signal. Exemplary embodiments of this signal path architecture include coaxial cable, microstrip, stripline, coplanar waveguide, slotline, and suspended substrate. Other exemplary transmission structures include wire, printed circuit board traces, conductive or dielectric waveguide structures, and mutlipolar (e.g., quadrapolar, octapolar) transmission structures. In one embodiment, the signal path includes a single signal port that receives an incident test signal and from which a reflected modulate signal is recovered. In another embodiment, the signal path consists of two or more signal ports: at least one that receives an incident test signal and one that outputs the corresponding modulated test signal.

As used herein, the term “electromagnetically coupled” refers to the transfer of detectable amounts of electromagnetic energy between two objects, e.g., the signal path and molecular events occurring with the test sample. The two objects can be electromagnetically coupled when the objects are in direct contact, (e.g., molecular events occurring along the surface of a microstrip transmission line), or when the objects are physically separated from each other (e.g., molecular events occurring within a sample which are separated from an open-ended coaxial probe by the wall of a PTFE tube). As a modification, the term “electromagnetically couples” will indicate the interaction of an electromagnetic signal (e.g., the incident test signal) with an object (e.g., molecular events occurring within the test sample).

As used herein, the term “detection region” refers to a region of the bioassay device in which the test sample and signal path are electromagnetically coupled. The detection region may be realized in a variety of forms, e.g., an area within a fluid transport channel located proximate to an open-ended coaxial probe, an area of a flow cell located within a waveguide aperture, or a region of PTFE tubing aligned between the transmission line and ground plane of a microstrip structure. The detection region is not limited to any particular volume, but is preferred to be less than 1 ml (1×10⁻⁶ m³) for most of the operations for which the present invention is primarily intended (detection of biologically important molecular events). Smaller detection region volumes such as 1 μl (1×10⁻⁹ m³), 1 nl (1×10⁻¹² m³), or 1 pl (1×10⁻¹⁵ m³) (or ranging anywhere there between) are even more preferable. The physical limits of a particular detection region are considered to be the limits of the space in which changes can be detected in a sample that is electromagnetically coupled to the signal path. These physical limits can be the result of physical structures (e.g., metal plates or screens that shield further sample from interacting with a signal) or simply be the detection limits for a particular signal path and detector in the presence of a volume of sample, not all of which couples in a detectable manner with the signal path.

As used herein, the term “test signal” refers to an AC signal. In specific embodiments, the test signal is preferably at or above 10 Hz and at or below 1000 GHz (1×10¹² Hz), such as 100 Hz, 1 KHz, 50 KHz, 500 KHz, 2 MHz, 10 MHz, 20 MHz, 45 MHz, 100 MHz, 200 MHz, 500 MHz, 1 GHz (1×10⁹ Hz), 2 GHz, 5 GHz, 7.5 GHz, 10 GHz, 12 GHz, 15 GHz, 18 GHz, 20 GHz, 25 GHz, 30 GHz, 44 GHz, 60 GHz, 110 GHz, 200 GHz, 500 GHz, or 1000 GHz and range anywhere there between. A preferred region is from 10 MHz to 40 GHz, a more particularly from 45 MHz to 20 GHz. “Test signal” can refer to a range of frequencies rather than a single frequency, and such a range can be selected over any terminal frequencies, including frequency ranges bounded by the specific frequencies named in this paragraph.

System

As used herein, the term “bioassay device” refers to a structure that incorporates the portion of the signal path operable to illuminate the supplied sample with an electromagnetic signal at the desired frequency of operation. In a preferred embodiment, the bioassay device further includes a cavity, recessed area, enclosure, tube, flow cell, or other surface feature or structure that is configured to retain a volume of sample within the detection region of the bioassay device. The bioassay device is not limited to any particular geometry or size, and is defined primarily by the architecture of the signal path and desired volume of the interrogated sample.

As used herein, the “bioassay system” refers to the bioassay device as described above, in combination with the components necessary to supply and recover the test signals to and from the bioassay device and to analyze the results therefrom. These components can include test equipment (e.g., a network analyzer, vector voltmeter, signal generator, frequency counter, spectrum analyzer), control equipment (e.g., computers, temperature compensation circuitry and components), and sample handling components.

As used herein, the term “matrix” or “binding matrix” refers to a layer of material on the bioassay device that is used as a spacer or to enhance surface area available for binding or to optimize orientation of molecules for enhanced binding, or to enhance any other property of binding so as to optimize the bio-assay device. The matrix layer can be formed from carbohydrates such as dextran, poly amino acids, cross-linked or non-cross linked proteins, and the like.

III. Signal Analysis Techniques

A number of signal analysis techniques are described in the detailed description below. For the detection of cellular events, any of these techniques can be used, as one is generally detecting any change in cellular activity as a result of the addition of a test substance to a cell medium (for unkowns tested for their ability to affect cells in general) or is comparing a signal for a test compound with a signal for a known compound using a know signal analysis system. Any change in the signal after the test substance is added is an indication of a change in cellular activity, if addition of the test substance is accounted for (relatively easily done in cellular systems, as cellular systems are much more easily analyzed than the molecular systems for which many of the apparatuses described herein were originally developed).

IV. Exemplary Molecular Detection Systems

Bioassay Test System

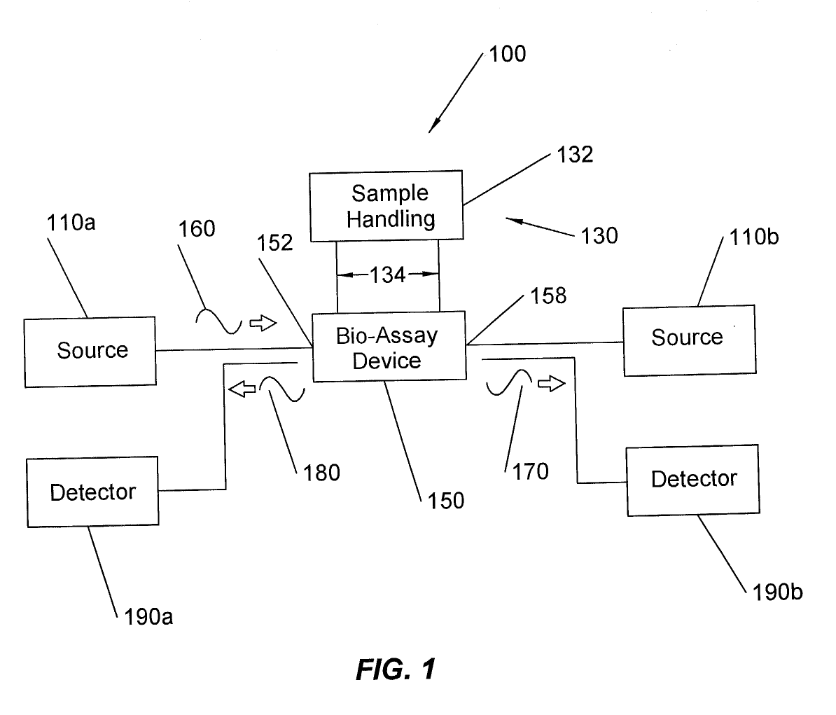

FIG. 1 illustrates a bioassay test system 100 in accordance with one embodiment of the detection system. The test system 100 includes a signal source 110a and a signal detector 190a connected to a first port of the biosensor 150. In this configuration, the signal source and detector can be used to obtain a one-port (i.e., a reflection) signal response. Alternatively, or in addition to the signal detector 190a, the test system 100 may include a signal detector 190b connected a second port 158 of the biosensor 150. When so configured, the signal source 110a and the signal detector 890b can be used to provide a two-port (i.e., a “through”) signal response of the biosensor 150. A second signal source 110b may be further included to provide a reflection measurement capability at the second port 158 of the biosensor 850.

The signal sources 110 are operable to generate and launch an electromagnetic signal 160 (“incident test signal”) at one or more amplitudes and/or frequencies. The signal detectors operate to recover the test signal after it has interacted with (i.e., after electromagnetically coupling to) the test sample in the biosensor 150. In a specific embodiment, the signal source 110 and the signal detector 190 are included within an automated network analyzer, such as model number 8510C from the Hewlett-Packard Company. Other measurement systems such as vector voltmeters, scalar network analyzers, time domain reflectometers, and the like that use signal characteristics of incident, transmitted, and reflected signals to evaluate an object under test may be used in alternative embodiment under the apparatus.

The sample handling assembly 130 includes a sample handling device 132 and a sample delivery apparatus 134. The sample handling device 130 may include sample preparation, mixing, and storage functions that may be integrated on a micro-miniature scale using, for instance, a microfluidic platform. The sample delivery apparatus 134 may consist of a tube, etched or photolithographcially formed channel or capillary, or other similar structure that delivers a volume of test sample to a location proximate to the signal path, such that the incident test signal propagating along the signal path will electromagnetically couple to the test sample. Specific embodiments of the sample handling and delivery structures are provided below.

The biosensor 150 operates as a bioelectrical interface that detects molecular events occurring within the sample using electromagnetic signals. The biosensor 150 includes a signal path that is configured to support the propagation of electromagnetic signals over the desired frequency range. Electrical engineers will appreciate that the signal path may consist of a variety of different architectures, for instance a waveguide, transverse electromagnetic (TEM) mode structures such as coaxial cable, coplanar waveguide, stripline, microstrip, suspended substrate, and slotline, as well as other structures such as twisted pair, printed circuits, and the like. Specific embodiments of the signal path are illustrated below.

An incident test signal 160 is generated by the signal source 110a and launched along the signal path where it electromagnetically couples from the signal path to the supplied test sample. One or more signal characteristics (amplitude, phase, frequency, group delay, etc.) of the incident test signal 160 are modulated by its interaction with the sample. In a one-port measurement system, a portion of the modulated signal 180 is reflected back along the signal path and recovered by the signal detector 190a. In a two-port measurement system, a portion of the modulated signal is transmitted through to the second port and recovered by the second signal detector 190b. The modulation caused by the electromagnetically coupling may consist of a change in the amplitude, phase, frequency, group delay, or other signal parameters.

The modulated test signal 180 (and/or 170) is recovered and its signal characteristics (amplitude, phase, etc.) are compared to signal characteristics of the corresponding incident test signal 160. In a particular embodiment, changes in the amplitude and phase of the modulated reflected signal 180 relative to the incident test signal 160 are computed at each test frequency and a response plotted over the test frequencies as an s-parameter return loss response. In another embodiment, changes in the amplitude and phase of the modulated transmitted signal 170 relative to the incident test signal 160 are computed at each test frequency and a response plotted over the test frequencies as an s-parameter transmission loss response. The signal responses may be used to compute other quantities to further characterize the test sample makeup. Quantities such as impedance, permeability, resonant frequency, and quality factor of resonant structures may also be either measured directly from the measurement system, or computed indirectly therefrom and used as a metric in characterizing the test sample.

Biosensors

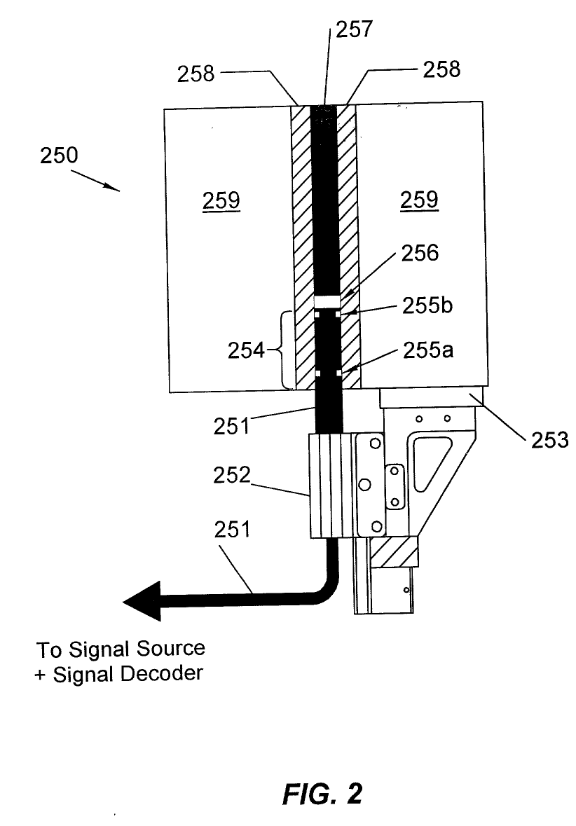

FIG. 2 illustrates a first embodiment of a biosensor, an open-ended coaxial resonant probe 250. The resonant probe 250 includes a first coaxial section 251, a bracket 252, an attachment platform 253, contact rings 255, a tuning gap 256, a second coaxial section 257, a conductive ground tube 258, and a fluidics shelf 259. The first coaxial section 251 is coupled to a signal source and a signal detector illustrated and described below. In one embodiment, the first and second coaxial sections consist of RG401 semi-rigid cable. Those of skill in the art will appreciate that other types of semi-rigid cable as well as other transmission structures can be used in alternative embodiments of the apparatus.

Securely held within the bracket 252, the first coaxial section 251 extends into the gap area 254 near the bottom of the fluidics shelf 259. Contact rings 255a and 255b can be optionally attached around the outer surface of the first coaxial section 251 to provide ground conductivity between the first coaxial section 251 and the inner surface of the ground tube 258. In one embodiment, the contact rings are highly conductive springs, although other structures can be used instead. In alternative embodiments, the outer surface of the first coaxial section 251 is brought into contact with the interior surface of the ground tube 258 (copper in one embodiment) to a sufficient degree, thereby obviating the need for the contact rings 255.

The second coaxial section 257 terminates in an open-end and has a length that is approximately one-half of a wavelength (λ/2) at the desired resonant frequency. In a specific embodiment, the first section 257 is approximately 4 inches, which corresponds to a resonant frequency of 1 GHz. The test sample is supplied at/near the open-end of the second coaxial section 257 such that a signal propagating along the second section 257 is electromagnetically coupled to the test sample. In one embodiment, the test sample comes into direct contact with the open-end cross-section of the second section 257. In another embodiment, the test sample and open-end section are separated by an intervening layer, such as the outer diameter of a fluidic channel or tube. In this instance, the intervening layer is sufficiently signal transparent to permit electromagnetic coupling through the intervening layer to the test sample. Occurrence of a molecular event may be detected either in “solid phase” by using probes immobilized over the detection region surface to bind to predefined targets in the solution, or in “solution phase” in which mobile molecular events occur over the detection region.

The first and second coaxial sections 251 and 257 are separated by a tuning gap 256 that electrically operates to fine-tune the resonant response to the desired frequency. In the illustrated embodiment, the second coaxial section 257 is secured within the ground tube 258 within the fluidics shelf 259. The first coaxial section 251 is inserted into the gap region 254, the outer surface of the first coaxial section 251 making electrical contact with the interior surface of the ground tube 258, thereby providing a continuous ground potential therebetween. The tuning gap 256 formed between the first and second coaxial sections 251 and 257 is made either shorter or longer by moving the bracket 252 either up or down, respectively. The reader will appreciate that the position of the second coaxial section 257 within the conductive ground tube 258 can be adjustable, either alternatively or in addition to the first coaxial section 251. The attachment platform 253 attaches to and holds stationary the fluidics shelf 259, allowing the bracket to either insert or remove the first coaxial section 251 therefrom. In a specific embodiment, the bracket 252 is motor driven and included within a precision motorized translational stage assembly available from the Newport Corporation of Irvine, Calif.

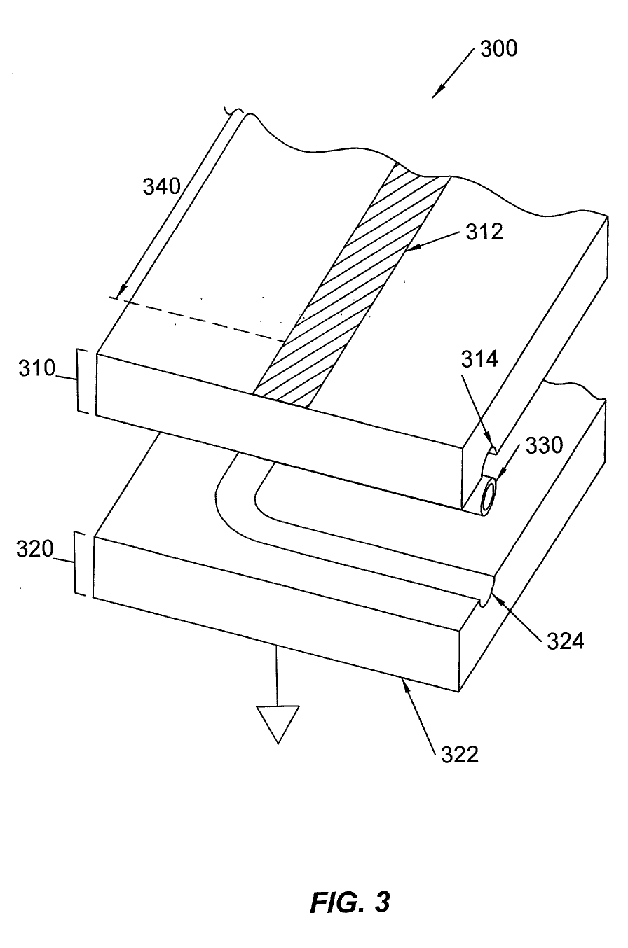

FIG. 3 illustrates a second embodiment of the biosensor, a broadband microstrip detector. The microstrip detector 300 includes top and bottom dielectric plates 310 and 320 and a flow tube 330 interposed therebetween. Top and bottom dielectric plates 310 and 320 are preferably constructed from a material exhibiting a low loss tangent at the desired frequency of operation. In the illustrated embodiment, the dielectric plates 310 and 320 are each 0.030″ thick of GML 1000 (available from Gil Technologies of Collierville, Tenn.) having a relative dielectric constant of approximately 3.2. In one embodiment, flow tube 330 is constructed from a material having a low loss tangent and a smooth, resilient surface morphology that inhibits analyte formation along the inner surface and detection of molecular events occur in solution phase as they move along the detection length 340 of the device. In another embodiment, the flow tube 330 may include immobilized probes on the inner surface that are operable to capture predefined targets occurring within the test sample. A PTFE tube having an ID of 0.015″ and OD of 0.030″ is used in the illustrated embodiment, although other materials and/or sizes may be used as well.

The top dielectric plate 310 includes a transmission line 312 deposited on the top surface and a channel 314 formed on the bottom surface. The width of transmission line 312 is chosen to provide a predetermined characteristic impedance along the detection length 340 (further described below). The impedance calculation may take into account the varying dielectric constants and dimensions introduced by channels 314 and 324 and flow tube 330. The transmission line 312 is typically formed from gold or copper.

The second dielectric plate 320 includes a channel 324 formed on the top surface and metallization deposited on the bottom surface. The channel 324 is aligned with channel 314 to form a cavity within which the flow tube 330 extends. The metallization 322 deposited on the bottom surface functions as the ground plane of the microstrip detector and will typically consist of a highly conductive material such as gold or copper. Channels 314 and 324 are aligned to form a cavity that retains the flow tube 330 in a substantially vertically aligned position between the transmission line 312 and the ground plane 322. The flow tube is held between the transmission line 312 and the ground plane 322 along the detection length 340. This configuration results in the passage of a significant number of field lines emanating from the transmission line through the flow tube (and accordingly, the test sample) before terminating on the ground plane 322. The dielectric properties of the molecular events within the sample will modulate the signal propagating along the transmission line 312 (i.e., by altering the field lines setup between the transmission line 312 and ground plane 322), thereby providing a means to detect and identify the molecular events occurring in the test sample.

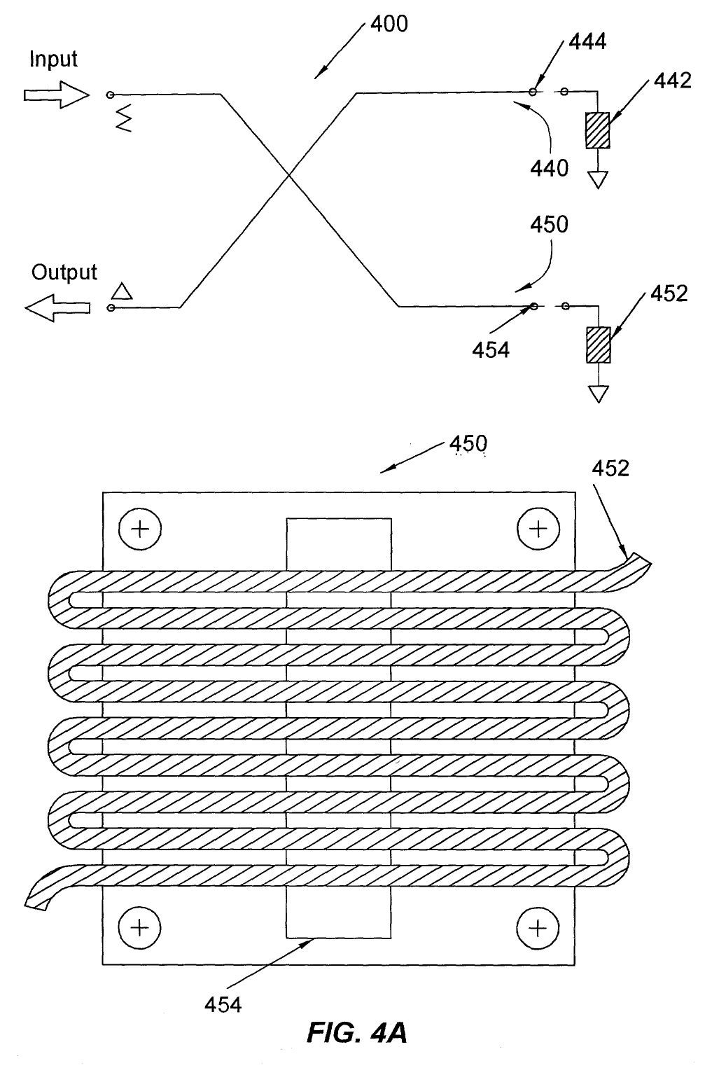

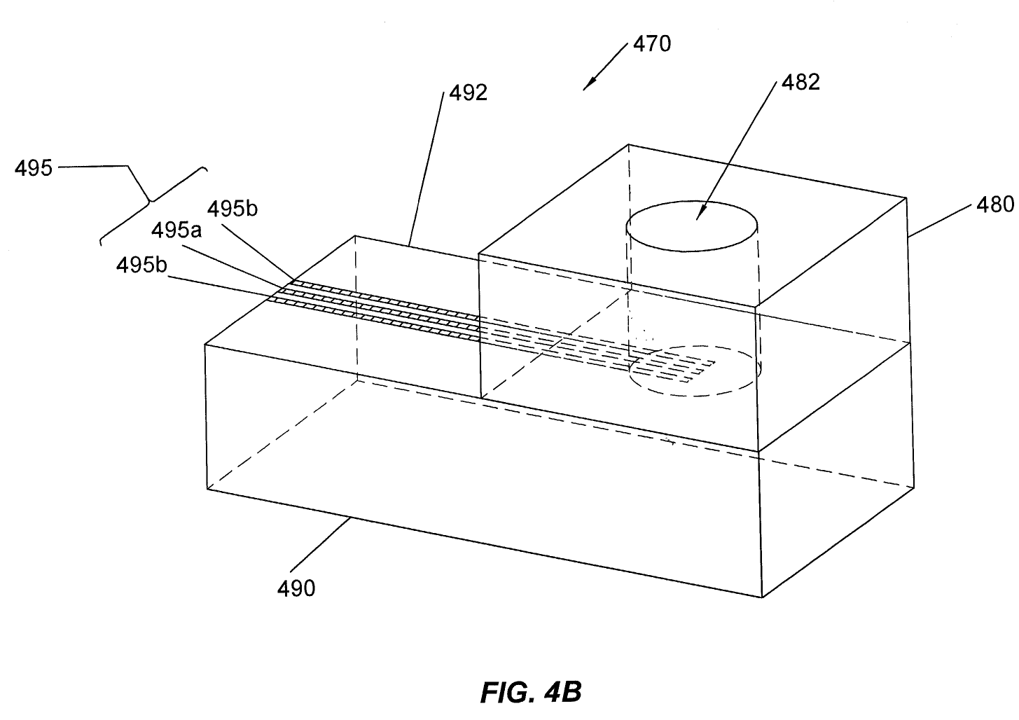

FIG. 4A illustrates a third embodiment of the biosensor, a waveguide magic-t coupler assembly. Known to practitioners in the area of high frequency circuit design, magic-t couplers can be configured to produce an output that represents the difference in the dielectric properties of two loads connected to the coupler. In the illustrated embodiment, two loads are connected to the magic-t coupler, the first load consisting of a reference sample in which a particular molecular event is known to be present or absent, and the second load consisting of an unknown sample that is being interrogated for the presence of the particular molecular event. A test signal at one or more frequencies is propagated into the structure and is electromagnetically coupled to the loads. The resulting output signal represents a comparison between the dielectric properties of the two loads.

The waveguide magic-t coupler includes two load ports (one shown in FIG. 4A) consisting of waveguide apertures over which a section of tubing 452 (PTFE in one embodiment) is meandered. Tubing 452 is operable to transport the sample to, and contain it within, a cross sectional area across the waveguide aperture 454 where the incident test signal electromagnetically couples to the sample. In a specific embodiment, the magic-t assembly consists of an X-band magic-t coupler (available from Penn Engineering North Hollywood, Calif.) and 0.020″ ID PTFE tubing.

FIG. 4B illustrates a fourth embodiment of the biosensor in accordance with the present invention, shown as a well-based coplanar waveguide transmission line. The biosensor 470 includes an upper substrate 480 and a lower substrate 490. The upper substrate 480 includes a cavity 482 extending through the depth of the upper substrate 480. The lower substrate 490 includes a top surface 492 onto which a center signal line 495a and two lateral ground elements 495b are formed. In another embodiment, an annular ring is used instead of the upper substrate 480. An additional ground plane may be additionally deposited on the bottom surface of the lower substrate 490. The signal transmission structure 495 may comprise a slot line structure in an alternative embodiment.

The upper substrate 480 is positioned on top of the lower substrate 490 and the two are aligned so that at least a portion of the signal transmission structure (the center signal line 495a and the ground elements 495b) extends into the cavity 482. The upper and lower substrates 480 and 490 are attached, thermally bonded by raising the glass near its softening point in one embodiment, to retain a predefined volume of a test sample within the cavity 482. In a specific embodiment, the upper substrate 480 and the lower substrate 490 are composed of borosilicate glass are attached by thermal bonding. The signal transmission structure 495 is formed by metal deposition using titanium or chrome adhesion layer (100 Å-200 Å) followed by a 1-2 um gold film and patterning using standard UV photolithography.

Sample Handling

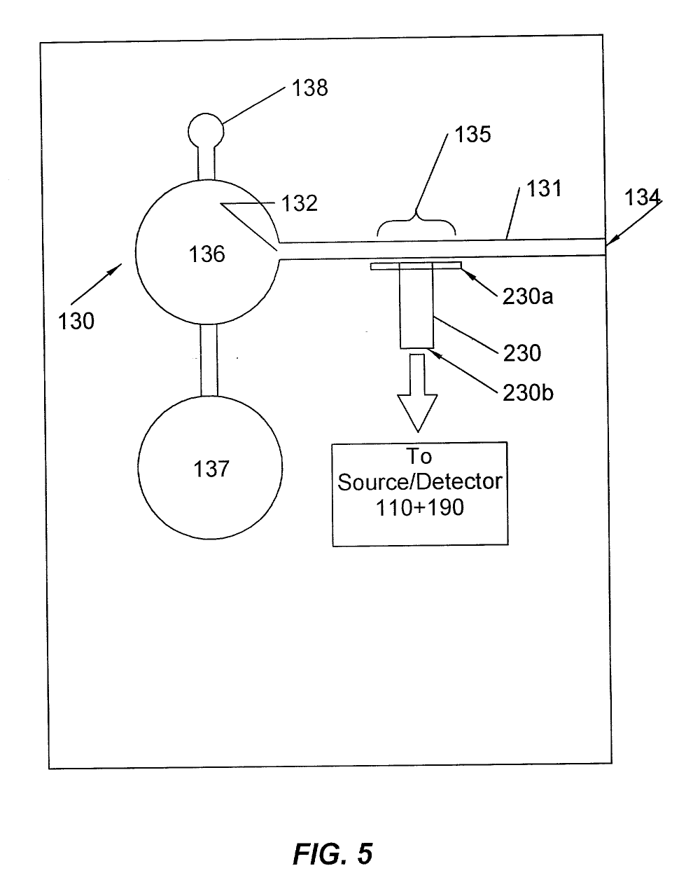

FIG. 5 illustrates a coaxial biosensor 230 integrated with a fluidic transport system 130 in accordance with one embodiment of the apparatus. The fluid transport system 130 includes a fluid channel 131 through which the test sample flows. Depending upon the application, the fluid channel 131 can take on a variety of forms. For instance in one embodiment, the fluid channel 131 is a Teflon® (polytetrafluoroethylene; PTFE) or other hard plastic or polymer tube (for example TEZEL™ (ETFE) tube) operable to transport the test sample to and from the detection region 131. In another embodiment, the channel 131 consists of one or more etched channels (open or enclosed) in a microfluidic transport system, further described below. Two or more channels can be used to provide a larger detection region 135 to improve detection sensitivity. In another embodiment, the fluid channel 131 is formed through well-known semiconductor processing techniques. Those of skill in the art will appreciate that other construction and architectures of the fluid channel 131 can be adapted to operate under apparatus.

The buffer can consist of a variety of solutions, gases, or other mediums depending upon the particular analyte therein. For example, when the detection system is used to detect the presence and/or binding of biological analytes, Dulbecco's phosphate buffer saline (d-PBS) or a similar medium can be used as a buffer to provide an environment which resembles the biological molecule's natural environment. As appreciable to those skilled in the art, other buffers such as DMSO, sodium phosphate (Na3PO4), MOPS, phosphate, citrate, glycine, Tris, autate, borate as well as others can be used in other embodiments.

The fluid channel 131 includes a detection region 135 over which the biosensor 230 illuminates the sample. Molecular event detection and/or identification can be accomplished in “solution phase” where the molecular events are free-flowing in the test sample as they move through the detection region, or alternatively in “solid phase,” in which probes are deposited or otherwise formed over the detection region and targeted molecular events attach thereto. The volume of the detection region 135 will be influenced by several factors including the architecture and material composition of the fluid channel 131, concentration of the molecular events occurring within the solution, desired detection time, the rate at which the test sample advances through the channel, and other factors as appreciable to those skilled in the art. In those embodiments in which detection occurs using immobilized probes, probes are formed within the detection region 135, the volume influenced by binding surface chemistry, the material and morphology of the binding surface, and other factors appreciable to those skilled in the art. Exemplary dimensions of the binding surface will be on the orders of 10⁻¹ m² to 10⁻¹⁵ m² or any range within these limits. The larger numbers in this range are preferably achieved in a small volume by using a convoluted or porous surface. Smaller numbers within those listed will be more typical of microfluidic devices and systems fabricated using semiconductor processing technology. The detection region 135 can alternatively be modified to accommodate other diagnostic applications, such as proteomics chips, known in the art. The size or shape of detection region need only be such that signal propagation thereto and analyte passage therethrough are possible, subject to other constraints described herein.

In the illustrated embodiment of the detector assembly 150, the fluid controller 136 is connected to a reservoir 137. Fluid controller 136 uses fluid from the reservoir 137 to move the test sample through channel 131, which requires less test sample than simple pumping of sample alone through the channel.

A second reservoir 138 can be used to store a second analyte or test sample for mixture with the reservoir 137 test sample. In such an embodiment, the fluid controller 136 can be further configured to rapidly mix the two test samples and supply the resulting mixture to the detection region 135. This technique (known as stopped-flow kinetics in the art of fluidic movement systems) permits the operator to observe and record changes in the signal response as binding events occur between analytes of the two test samples. This data can also be used to determine the kinetics of binding events occurring between the analytes of the two samples. The fluidics of conventional stopped-flow kinetic systems, such as model no. Cary 50 available from Varian Australia Pty Ltd. of Victoria, Australia, can be adapted to operate with the apparatus or integrated within the detector assembly 150. See www.hi-techsci.co.uk/scientific/index.html for additional information about stopped-flow fluidic systems.

Other components can be included to regulate the test sample flow through the channel 131. The fluid controller 136, fluid reservoirs 137 and 138 and other components associated with fluidic movement can comprise discrete components of the fluid transport system 130 or alternatively be partially or completely integrated.

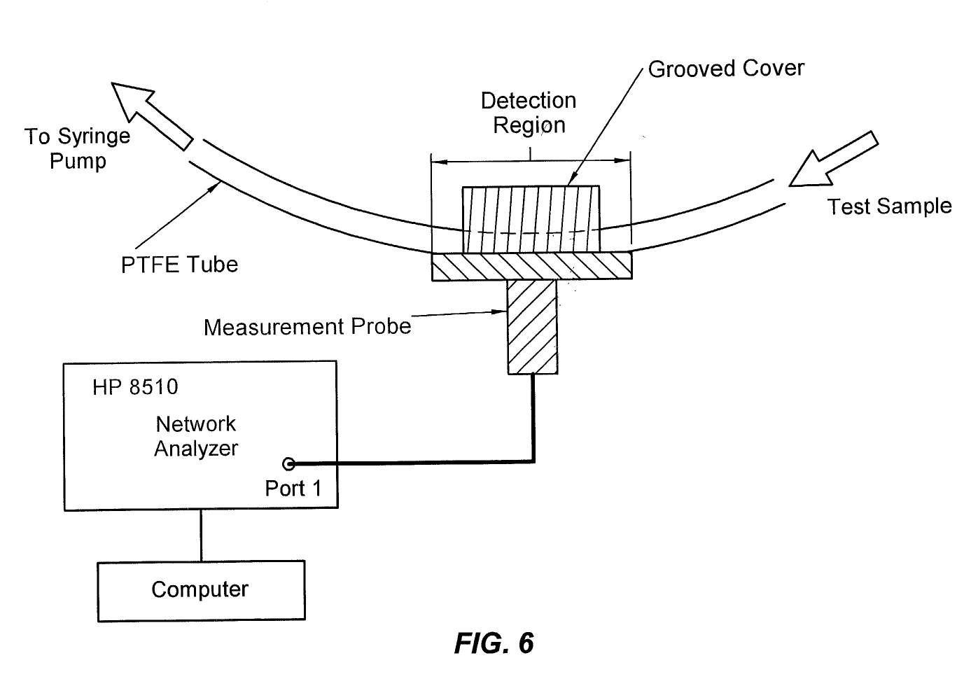

FIG. 6 illustrates a bioassay test system in which a flow tube is used to supply the sample to a coaxial probe. The system includes a vector network analyzer model number HP 8714 available from Agilent Technologies, Inc. (formerly the Hewlett Packard Corporation), a computer, an open-ended coaxial measurement probe flunctioning as the biosensor, and a length of PTFE tube (Cole-Parmer Instrument Company of Vernon Hills, Ill.) used as a fluid channel to transport the transporting medium and test sample to the detection region of the measurement probe. The PTFE tube (0.031″ I.D., 0.063″ O.D., wall 0.016″) is located over the detection region of the measurement probe and is secured using a grooved top cover that was screwed into the shelf of the measurement probe. The tubing extends from the measurement probe in two directions. One end of the tubing is connected to a syringe pump while the other end was immersed in the fluidic test sample to be analyzed. The syringe pump provided negative pressure that was applied to pull the test sample through the tube and over the detection region. In a specific embodiment, the syringe pump aspirates fluid at a rate of ˜0.05 mL/min. Further preferred is the introduction of air gaps between two test samples to prevent mixing.

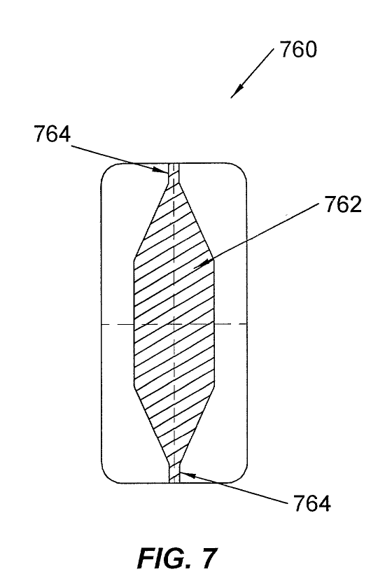

FIG. 7 illustrates a flow cell 760 for use with the waveguide magic-t detector shown in FIG. 4. The flow cell 760 is sized to fit into the waveguide aperture 454 located at the load ports and is constructed from acrylic ([poly]methylmethacrylate) in one embodiment. The flow cell 760 includes a sample chamber 762 (holding 25 μl in one embodiment) and inlet/outlet needles 764, which are UV epoxied to the ends of the chamber 762. Preferably, the diameter of needles 764 is chosen to insert securely within a section of tubing (0.020″ ID PTFE tube in one embodiment) which supplies the sample.

V. Exemplary Application Methods

The apparatuses and sub-assemblies described herein can used to provide information about numerous properties of a test sample, such as the detection and identification of molecular binding events, analyte concentrations, changes in dielectric properties of the bulk test sample, classification of detected binding events, and the like. Each of these methods and capabilities are further described below. Based upon the described methods and structures, modifications and additional uses will be apparent to those skilled in the art.

The herein-described systems and methods can be used in a variety of analytical applications. In one embodiment, the systems can be used in methods that identify substructures or binding events involving analytes, for example proteins. In a calibration phase of such analyses, the signal responses of a large number of known proteins can be determined and stored. After introducing an unknown protein to the detection region, the dielectric properties of the system can be measured and the dielectric properties of the signal used to identify the protein's properties. Because each protein's fingerprint response is stored, the unknown response can be compared with the stored responses and pattern recognition can be used to identify the unknown protein.

In another embodiment, the invention can be used in a parallel assay format. The device in such a format will have multiple addressable channels, each of which can be interrogated separately. After delivering a test sample or samples to the device, responses at each site will be measured and characterized. As an example, a device of this type can be used to measure and/or identify the presence of specific nucleic acid sequences in a test sample by attaching a unique nucleic sequence as the antiligand to the detection region or a part thereof. Upon exposure to the test sample, complementary sequences will bind to appropriate sites. The response at each site will indicate whether a sequence has bound. Such measurement will also indicate whether the bound sequence is a perfect match with the antiligand sequence or if there are one or multiple mismatches. This embodiment can also be used to identify proteins and classes of proteins, by analyzing signals obtained from a particular sample and comparing that signal to signals obtained from a collection of known proteins.

In another embodiment, the present invention can be used as part of a technique that generates a standard curve or titration curve that would be used subsequently to determine the unknown concentration of a particular analyte or ligand-binding curve. For example, an antibody could be attached to the detection region. The device could be exposed to several different concentrations of the analyte and the response for each concentration measured. Such a curve is also known to those skilled in the art as a dose-response curve. An unknown test sample can be exposed to the device and the response measured. Its response can be compared with the standard curve to determine the concentration of the analyte in the unknown test sample. Similarly, binding curves of different ligands can be compared to determine which of several different ligands has the highest (or lowest) affinity constant for binding to a particular protein or other molecule.

In another embodiment, this invention can be used with embodiments that calibrate for losses due to aging and other stability issues. For example with antibody-antigen systems, one can measure the amount of active antibody in a test sample. The signal response is compared to standard signals for samples of known activity in order to determine the activity of the unknown.

Detecting Cellular Activity and Cellular Events

The general technology developed in the laboratories of the inventors is often referred to as multipole coupling spectroscopy (MCS) and is a variation of dielectric spectroscopy. Prior to the present work, most developments in the dielectric spectroscopy area had taken place at frequencies below 10 MHz and often even lower. For example, U.S. Pat. No. 5,187,096 describes a “Cell Substrate Electrical Impedance Sensor with Multiple Electrode Array” as using an AC current of about one microamp and fixed or varying frequencies, without specifically specifying the frequency range. However, the only frequencies shown in the examples are in the range from 1 Hz to 100 KHz, and the electrical architecture of the apparatus as shown is more appropriate for this lower frequency range than for higher ranges.

While MCS can be used in these lower frequencies, MCS is typically used in the detection of protein soft vibrations involving protein motions in the 10 picosecond to 100 nanosecond range, as well as various molecular events of the type described in more detail in earlier applications of this series (q.v.). The present invention, rather than being specifically directed to those molecular events, focuses on detection of cellular events (cellular activity), whether as the result of some natural process occurring within the cell (without external intervention) or as the result of addition of a test compound to a composition containing a cell or cells in order to determine the manner in which that test compound enhances, inhibits, or otherwise modifies cellular activity. These detection processes can take place in solution or on surfaces (solid-phase detection), as described previously in detail for molecular events.

One of the areas in which MCS can provide a number of advantages when used to detect cellular activity and events is in drug discovery. Many steps of a typical drug-discovery process (e.g., hit detection, lead discovery, or lead optimization) can be aided by using cellular activity analysis in addition to (or in place of) assays that detect only cell-free molecular binding interactions. Molecular interaction detection is typical of the earlier stages of drug discovery (e.g., target identification, hit detection and lead discovery), and MSC has demonstrated the ability to identify proteins as having similar structures (target detection) and the ability to qualitatively and quantitatively measure molecular interactions (hit detection). Cellular activity becomes more important at the lead discovery stage, as one becomes more concerned with the activity of the cell as a whole rather than simply with some desired level of molecular activity. At the level of lead optimization (structure/activity relationships), cellular systems come to the forefront, as the need to determine all possible actions of a lead compound become important, including actions induced by the lead compound in parts of the cell that are not part of the specific molecular target involved in the molecular interaction used to identify the lead compound.

This is not to say, however, that molecular interactions are not taking place. Indeed, molecular interactions are the principal mechanism through which cell activity is triggered. One advantage of working with cellular systems (as opposed to the molecular systems that have been the principal target of earlier operations in the laboratories of the inventor) is the inherent amplification that takes place in cellular systems as the result of enzymatic and other types of cellular processes. For example, instead of detecting the molecular interaction of a ligand with a receptor in a cell-free system, the ligand can induce cellular activity that results in major chemical or morphological changes in a cell. Examples of cellular activity that can be triggered by an initial molecular interaction (on the surface or in the cytoplasm or nucleus of a cell) include GPCR-mediated pathway induction, ion-channel modulation, morphologic changes, apoptotic events, cytosolic cAMP/Ca ion events, membrane changes, and protein expression levels. These cellular events can be detected via a spectral response at one or more frequencies or via cellular kinetics, and the ability of a test substance to affect the properties of cells of different types can be compared using genetically modified cells or other diverse cellular populations.

The invention provides cell-based assays that do not require sample purification or amplification. In these cell-based assays, cellular systems can be monitored for changes, indeed, any change in cellular activity such as an increase (or decrease) in the amount of any detectable cellular constituent (e.g., a protein, a lipid, a carbohydrate, a nucleic acid, water [cell volume], or an ion [ion influx]). For example, one can detect expression as a result of increased cellular activity in the presence of an inducer.

Accordingly, the present invention provides target identification and validation, rapid assays, secondary screening, and lead optimization in a homogeneous assay without use of radioactivity or other types of labels.

VI. Software Implementation

Each of the measurement and detection methods described herein can be practiced in a multitude of different ways (i.e., software, hardware, or a combination of both) and in a variety of systems. In one embodiment, the described method can be implemented as a software program.

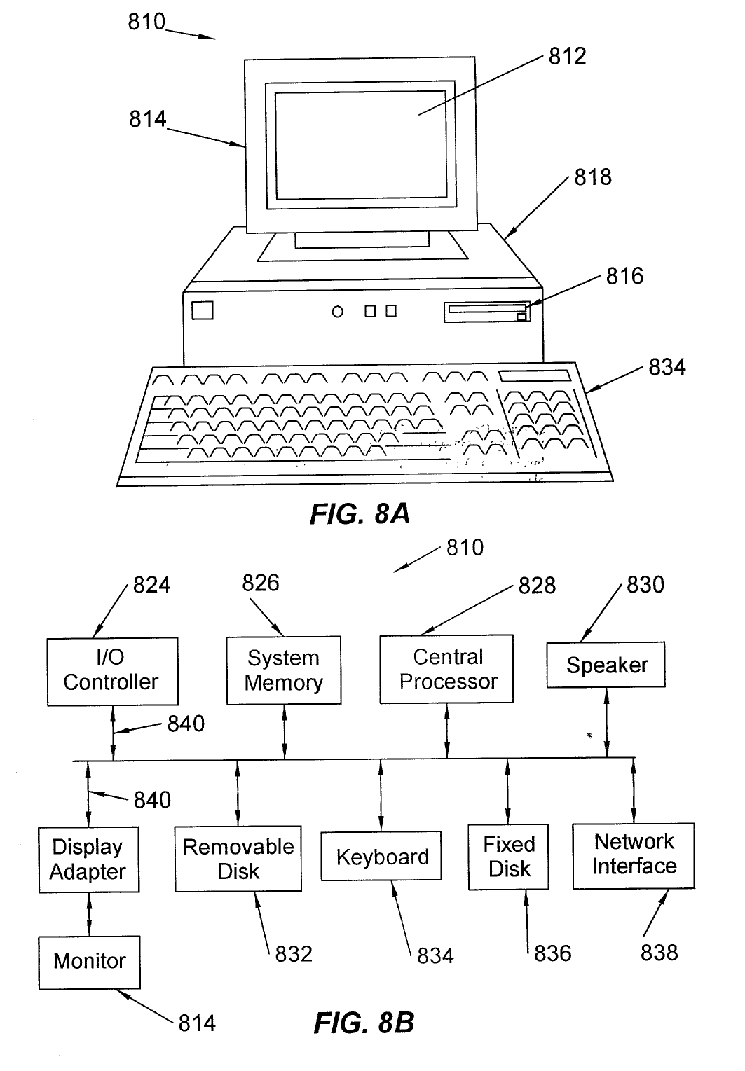

FIG. 8A illustrates a simplified block diagram of a computer system 810 operable to execute a software program designed to perform each of the described methods. The computer system 810 includes a monitor 814, screen 812, cabinet 818, and keyboard 834. A mouse (not shown), light pen, or other I/O interface, such as virtual reality interfaces can also be included for providing I/O commands. Cabinet 818 houses a CD-ROM drive 816, a hard drive (not shown) or other storage data mediums which can be utilized to store and retrieve digital data and software programs incorporating the present method, and the like. Although CD-ROM 816 is shown as the removable media, other removable tangible media including floppy disks, tape, and flash memory can be utilized. Cabinet 818 also houses familiar computer components (not shown) such as a processor, memory, and the like.

FIG. 8B illustrates the internal architecture of the computer system 810. The computer system 810 includes monitor 814, which optionally is interactive with the I/O controller 824. Computer system 810 further includes subsystems such as system memory 826, central processor 828, speaker 830, removable disk 832, keyboard 834, fixed disk 836, and network interface 838. Other computer systems suitable for use with the described method can include additional or fewer subsystems. For example, another computer system could include more than one processor 828 (i.e., a multi-processor system) for processing the digital data. Arrows such as 840 represent the system bus architecture of computer system 810. However, these arrows 840 are illustrative of any interconnection scheme serving to link the subsystems. For example, a local bus could be utilized to connect the central processor 828 to the system memory 826. Computer system 810 shown in FIG. 8B is but an example of a computer system suitable for use with the present invention. Other configurations of subsystems suitable for use with the present invention will be readily apparent to of skill in the art.

VII. Example of Cellular Activity Detection

Ability of MCS to detect cellular activity has been demonstrated using a number of model systems. For example, the muscarinic m1 receptor that had been transfected into CHOk1 cells (Chinese Hampster Ovary wild-type cells) to form CHOm1 (transfected) cells was activated in the presence of agonists, and activation was inhibited in the presence of antagonists. In one such assay, CHOm1 and CHOk1 cells were treated with carbachol, a known activator of the m1 receptor. Differential activation of these cell types can readily be seen using the well-known LJL fluorescent assay, and this differentiation of activity could be detected using MCS. There was some variation from day to day in the common manner that occurs with cellular assays; most of this difference appeared to be related to differences is cell health or activity state following cell division. The assays were carried out using a coplanar waveguide apparatus described in detail in a co-filed application filed on even date herewith (entitled “Coplanar Waveguide Biosensor for Detecting Molecular or Cellular Events;” Attorney Docket No. 25US). Measurements were made over the range from 50 MHz to 1 GHz using SP21 (a two-port biosensor for measuring transmission loss) and SP11 (a one-port biosensor for measuring return loss) detectors, both gold and platinum chips, with 5×10⁴ cells/well plated the day before and using a sucrose buffer containing 1.26 mM CaCl₂, 0.81 mM MgSO₄, 5.37 mM KCl, 1 mM MgCl₂, 5 mM NaCl, 10 mM Hepes, 16 mM glucose, and 230 mM sucrose.

In a first series of measurements, taken 7 minutes after addition of carbachol and plotted over the indicated frequency range, un-transfected cells (CHOk1) were similar to controls, while transfected (CHOm1) cells treated with 10 uM carbachol showed a significant change in signal at all frequencies over the tested range. When the same assay was carried out in the presence of the antagonist pirenzepine, cells treated with agonist alone were significantly different from controls, while the cells that had been blocked by pre-treatment with 1 uM pirenzepine were similar to controls.

Since the differences were found to be proportional over the measurement range used, many further measurements were made at a single frequency (107 MHz) in order to carry out the assay more rapidly without recording an entire spectrum. In one such assay, transfected cells treated with 10 uM agonist were significantly different from both wild-type cells treated in the same manner and transfected cells that had been pretreated with 1 uM antagonist (the latter two being very similar to each other).

Other types of signal analysis gave similar results. In some cases, the signal over the 50 MHz to 1 GHz range was integrated and the resulting integral for the signal of a test compound/cellular system was compared to the integrated signal for a buffer/cellular system. In one such assay, CHOk1 cells treated with various concentrations of carbachol showed essentially no change in signal over time, while CHOm1 transfected cells showed a typical dose/response effect of increased activity with time after addition of various concentrations of carbachol. Using the same integrated signal analysis technique, CHOm1 cells treated with 300 nM carbachol and varying amounts of the inhibitor pirenzepine showed a typical dose response for the inhibitor, with higher concentrations of the inhibitor returning the response curve to levels of unactivated cells.

Using a different method of signal analysis (determining the increase in slope of the signal from before to after treatment 300 nM carbachol after pre-treatment with different levels of the inhibitor pirenzepine), transfected cells showed similar activity in both the standard LJL (calcium fluorescence) measurement used as a control assay and a MCS assay as described herein, thus verifying the ability of the assay of the invention to replicate a known assay, but without requiring the addition of fluorescent dyes or other materials that may affect cellular activity in unexpected ways. Thus, the assay has been verified, although significant daily variation can occur (as is typical of cellular systems).

The assay was repeated with a different inhibitor (telenzepine), and a similar dose/response curve was obtained using different inhibitor concentrations and 1 uM carbachol. As with earlier assays, differences in cellular activity were detectable in the first minute after addition of carbachol. The two inhibitors, however, produced different dose/response curves, and a plot of antagonist concentration versus assay result (in this case plotted as the slope of the integral change) clearly demonstrated the relative inhibition abilities of the two antagonists.

Ability to detect other calcium flux modifies was tested using ionomycin (an ionophore). In this case both wild-type and transfected cells showed similar results, as the change in cellular activity was not mediated by a specific receptor, but by a non-specific ionophore. Ability of the ionophore inhibitor thapsigargin to inhibit ionomycin was then demonstrated using the MCS system.

While the above is a complete description of possible embodiments of the invention, various alternatives, modifications, and equivalents can be used. For example, other transmission mediums, such as conductive or dielectric waveguides, can alternatively be used, as well as other fluid transport systems. Further, all publications and patent documents recited in this application are incorporated by reference in their entirety for all purposes to the same extent as if each individual publication and patent document was so individually denoted. Specifically, this application is related to the following commonly owned, co-pending applications, all of which are herein incorporated by reference in their entirety for all purposes:

Ser. No. 09/243,194 entitled “Method and Apparatus for Detecting Molecular Binding Events,” filed Feb. 1, 1999 (Atty Dkt No. 19501-000200US);

Ser. No. 09/365,578 entitled “Method and Apparatus for Detecting Molecular Binding Events,” filed Aug. 2, 1999 (Atty Dkt No. 19501-000210);

Ser. No. 09/243,196 entitled “Computer Program and Database Structure for Detecting Molecular Binding Events,” filed Feb. 1, 1999 (Atty Dkt No. 19501-000300);

Ser. No. 09/480,846 entitled “Resonant Bio-assay Device and Test System for Detecting Molecular Binding Events,” filed Jan. 10, 2000 (Atty Dkt No. 19501-000310);

Ser. No. 09/365,978 entitled “Test Systems and Sensors for Detecting Molecular Binding Events,” filed Aug. 2, 1999 (Atty Dkt No. 19501-000500);

Ser. No. 09/365,581 entitled “Methods of Nucleic Acid Analysis,” filed Aug. 2, 1999 (Atty Dkt No. 19501-000600);

Ser. No. 09/365,580 entitled “Methods for Analyzing Protein Binding Events,” filed Aug. 2, 1999 (Atty Dkt No. 19501-000700);

Ser. No. 09/687,456 entitled “System and method for detecting and identifying molecular events in a test sample,” filed Oct. 13, 2000 (Atty Dkt No. 12US);

Serial No. 60/248,298 entitled “System and method for real-time detection of molecular interactions,” filed Nov. 13, 2000 (Atty Dkt No. -14P);

Ser. No. 09/775,718 entitled “Biosensor device for detecting molecular events,” filed Feb. 1, 2001 (Atty Dkt No. -15US);

Ser. No. 09/775,710 entitled “System and method for detecting and identifying molecular events in a test sample using a resonant test structure,” filed Feb. 1, 2001 (Atty Dkt No. -16US);

Serial No. 60/268,401 entitled “A system and method for characterizing the permittivity of molecular events,” filed Feb. 12, 2001 (Atty Dkt No. -17P);

Ser. No. 60/275,022 entitled “Method for detecting molecular binding events using permittivity,” filed Mar. 12, 2001 (Atty Dkt No. -18P);

Serial No. 60/277,810 entitled “Biosensor device for Detecting Molecular Events,” filed Mar. 21, 2001 (Atty Dkt No. -19P);

Ser. No. 09/837,898 entitled “Method and Apparatus for Detection of Molecular Events Using Temperature Control of Detection Environment,” filed Apr. 18, 2001 (Atty Dkt No. -20US);

Ser. No. 09/880,331 entitled “Reentrant Cavity Biosensor for Detecting Molecular Events,” filed Jun. 12, 2001 (Atty. Dkt. No. -21US); and

Ser. No. 09/880,746 entitled “Pipette-Loaded Biosensor Assembly for Detecting Molecular Events,” filed Jun. 12, 2001 (Atty Dkt. No. -22).

The following commonly owned applications are concurrently filed herewith, and are incorporated by reference in their entirety for all purposes:

“Well-based Biosensor for Detecting Molecular or Cellular Events,”(Atty. Docket No. 24 US); and

“Coplanar Waveguide Biosensor for Detecting Molecular or Cellular Events,” (Atty. Docket No. 25 US).

What is claimed is:

1. A method of assaying cellular activity by monitoring a change in a cellular system, comprising:
coupling an electromagnetic test signal in a frequency range from 10 MHz to 1000 GHz to a sample in which a cellular event is being detected, whereby said sample interacts with and modulates said test signal to produce a modulated test signal;
detecting said modulated test signal; and
analyzing said modulated test signal to detect said cellular event.

2. The method of claim 1, wherein said cellular activity comprises a change in amount of a substance present in said cell as the result of presence of a test substance in a medium containing said cell.

3. The method of claim 1, wherein said substance is a protein, a lipid, a carbohydrate, a nucleic acid, water, or an ion.

4. The method of claim 1, wherein said cell comprises artificially inserted genetic material encoding a target receptor.

5. The method of claim 1, wherein said cell is a wild-type cell.

6. The method of claim 2, wherein said cell comprises a receptor having a known activity and said change results from activity of said test substance as an agonist or antagonist of said receptor activity.

7. The method of claim 1, wherein said change is opening or closing of an ion channel.

8. The method of claim 1, wherein said cell is a mammalian cell.

9. The method of claim 8, wherein said cell is a CHO cell.

10. The method of claim 1, further comprising verifying said method by correlating with a known cell activity of a known substance prior to testing an unknown substance.

11. A method of assaying cellular activity by monitoring a change in a cellular system, comprising:
coupling an electromagnetic test signal to a sample in which a cellular event is being detected, whereby said sample interacts with and modulates said test signal to produce a modulated test signal;
detecting said modulated test signal; and
analyzing said modulated test signal to detect said cellular event,
whereby said sample is coupled to said signal by a one-port coplanar waveguide transmission line operable to support the propagation of a electromagnetic test signal, comprising:
a signal line configured to conduct a time-varying voltage therealong; and
one or more ground elements configured to maintain a time-invariant voltage therealong, the one or more ground elements spaced apart from the signal line and located generally within the same plane as the signal line, wherein a detection region is formed between a portion of the signal line and a portion of at least one of the one or more ground elements; and
whereby said sample is contained in a sample containment structure intersecting the detection region of the one-port coplanar waveguide transmission line, wherein the sample containment structure comprises a cavity operable to hold 1 ml or less of sample solution within the detection region.

12. The method of claim 11, wherein said cellular activity comprises a change in amount of a substance present in said cell as the result of presence of a test substance in a medium containing said cell.

13. The method of claim 11, wherein said substance is a protein, a lipid, a carbohydrate, a nucleic acid, water, or an ion.

14. The method of claim 11, wherein said cell comprises artificially inserted genetic material encoding a target receptor.

15. The method of claim 11, wherein said cell is a wild-type cell.

16. The method of claim 12, wherein said cell comprises a receptor having a known activity and said change results from activity of said test substance as an agonist or antagonist of said receptor activity.

17. The method of claim 11, wherein said change is opening or closing of an ion channel.

18. The method of claim 11, wherein said cell is a mammalian cell.

19. The method of claim 18, wherein said cell is a CHO cell.

20. The method of claim 11, further comprising verifying said method by correlating with a known cell activity of a known substance prior to testing an unknown substance.
